(19) United States
(12) Patent Application Publication
(10) Pub. No.: US 2005/0266403 A9
(43) Pub. Date: Dec. 1, 2005

(54) Method for analyzing base sequence of nucleic acid

(76) Inventors: Nobuko Yamamoto, Kanagawa (JP); Tadashi Okamoto, Kanagawa (JP); Tomohiro Suzuki, Kanagawa (JP)

Correspondence Address: FITZPATRICK CELLA HARPER & SCINTO, 30 ROCKEFELLER PLAZA, NEW YORK, NY, 10112, US

(21) Appl. No.: 10/231,302

(22) Filed: Aug. 30, 2002

Related U.S. Application Data
(63) Continuation of application No. PCT/JP00/07244, filed on Oct. 18, 2000.

(51) Int. Cl. C12Q 1/68; C07H 21/04

(52) U.S. Cl. 435/6; 536/24.3

(57) ABSTRACT

Provided is a method for performing a hybridization reaction that comprises the steps of providing a sample containing a target single-stranded nucleic acid and a probe array; heat-denaturing the probe array in a solution containing the sample; and reducing temperature to the extent suitable for a double-strand formation reaction, wherein the probe array remains immersed in the sample solution during reducing the temperature. Also disclosed is a method for detecting a certain sequence in a sample.

This application is a continuation of International Application No. PCT/JP00/07244 filed on Oct. 18, 2000.

### BACKGROUND OF THE INVENTION

1. Field of the Invention

The present invention relates to a method for determining the base sequence of a nucleic acid by using a DNA chip for DNA diagnosis and treatment.

2. Related Background Art

One of the techniques for sequencing nucleic acid etc. or for detecting the sequence is to utilize a DNA array. U.S. Pat. No. 5,445,934 discloses a DNA array where 100,000 or more oligonucleotide probes are bonded in 1-inch square. Such a DNA array has an advantage that many characteristics can be examined at the same time with a very small sample amount. When a fluorescence-labeled sample is poured onto such a DNA chip, DNA fragments in the sample bind to probes having a complementary sequence fixed on the DNA chip, and only that part can be discriminated by fluorescence to elucidate the sequence of the DNA fragment in the DNA sample.

Sequencing By Hybridization (SBH) is a method for examining the base sequence utilizing such a DNA array and the details are described in U.S. Pat. No. 5,202,231. In the SBH method, all possible sequences of an oligonucleotide of a certain length are arranged on the substrate, then fully matched hybrids formed by hybridization reaction between probes and the sample DNA are detected. If a set of fully matched hybrids is obtained, the set will give an assembly of overlapping sequences with one base shift being a part of one certain sequence, of which analysis will elucidate that sequence.

In principle, in order to examine whether a certain sequence is present or not in a DNA specimen, hybridization reaction is carried out with a prove having a complementary sequence, and the presence or absence of hybridization is detected. In practice, however, it is very difficult to judge the presence or absence of one sequence by using one complementary probe and hybridization, because even when fully matched hybrids are compared, the fluorescence intensities of the hybrids differ from each other according to their sequence. In particular, GC content in the sequence greatly affects the stability of the hybrid. Further, sequences not exactly complementary but containing one base mismatch also form a hybrid to emit fluorescence. Such a hybrid has lower stability and weaker fluorescence compared with a fully matched hybrid of the same sequence, but it is often observed that such a mismatch hybrid has a stronger fluorescence than a full-matched hybrid of different sequence. In addition, the stability of one mismatch hybrid greatly varies according to the location of the mismatch in the hybrid. When the mismatch locates at the terminus, a relatively stable hybrid is obtained. When the mismatch locates at the center of the hybrid, the hybrid becomes unstable because the consecutiveness of the complementary strand is broken. Thus, at present, various factors are complexly participating in the hybrid stability, and the absolute value (standard value) for the fluorescence intensity to judge whether the hybrid is fully matched or not is not obtained. Also, conditions for detecting the fluorescence solely from the fully matched hybrid, eliminating fluorescence from one-base mismatched hybrids have not been determined.

In order to dissolve the difference of the hybrid stability due to the sequence, a method using tetramethylammonium chloride is described in Proc. Natl. Acad. Sci. USA Vol. 82, pp. 1585-1588 (1985). However, the above-described problems have not been solved perfectly.

A method for judging whether a hybrid being a perfect match is described in Science vol. 274 p. 610-614, 1996, in which a 15-mer oligonucleotide probe and 15-mer oligonucleotides having the same sequence except for one mismatching base at the center of the sequence are prepared, and the fluorescence intensity of the hybrid with the probe (perfect match) is compared with those of hybrids with other one-base mismatching oligonucleotides, and only when the intensity of the perfect match is stronger, it is judged positive.

Based on the method above, U.S. Pat. No. 5,733,729 discloses a method using a computer for more accurate calling, where the fluorescence intensities of the hybrids are compared by using a computer to know the base sequence of a sample.

In these methods, it is necessary to locate the subject nucleotide to be examined in the center of a probe and to prepare a set of four probes each having one of four bases at the position. It is also necessary to prepare such a probe set for each of overlapping sequences with one base shift. As described above, they use 15-mer oligonucleotides and determine the perfect match by comparing with other three types of probes having one-base mismatch at the center. It is said that more accuracy can be obtained by evaluating the stability of the hybrids theoretically or empirically. In addition, if the base length of the region to be examined is L, the number of probes will be 4×L (e.g., 20 probes for 5 bases).

Although the above described methods using mismatches are excellent in that the call is relatively easy by comparing with one-base mismatches at the same position of the same sequence and that the number of probes may be small (in SBH, 1024 types of probes are required for the similar analyses), they have significant defects that accurate information can not be obtained when there are two base mismatches in the same region or when there is a base deletion or insertion.

On the other hand, the SBH method may solve the above-described problems and in principle, it may cope with any variation. Call, however, is rather difficult, since intensity of one-base mismatch in one sequence is stronger than that of a full match in another sequence and since stability of the hybrid differs considerably according to the position of the mismatch in the sequence even if it is a one-base mismatch. As a result, a full match, one-base and two-base mismatches (continuous or discontinuous) cannot be simply called from the fluorescence intensities. Accordingly complex analyses including theoretical predictions, comparison between individual sequences and accumulation of empirical parameters are required.

Furthermore, in order to determine the sequence of a gene by reading fluorescence intensities of hybrids for each probe followed by data analysis, a large-scale computer system as well as a detector for reading arrays is required. This is a big obstacle in the way of simple gene diagnosis using the DNA array.

In view of such problems, the present invention provides a method of accurate gene sequencing not requiring complex analyses.

### SUMMARY OF THE INVENTION

As described above, the fluorescence intensity of a hybrid is controlled by various factors. Thus, when a probe having about 15 mer to 20 mer in length is used, it is hard to exclude the fluorescence due to hybrids having one-base mismatch. On the other hand, it is relatively easy to obtain the conditions for inhibiting formation of two-base mismatch hybrids regardless of position, and continuity or discontinuity of the two-base mismatch.

The present invention has been achieved based on such a finding characterized in that spots of mismatch hybrids containing a predetermined number of mismatches are taken into account as well as a spot of a perfect match hybrid.

That is to say, one embodiment of the present invention is a method for determining an unknown base sequence in a given region of a target single-stranded nucleic acid which comprises the following steps (a) to (e):

(a) preparing a probe array in which a first to nth (n≧2) single-stranded nucleic acid probes having base sequences respectively complementary to every base sequence expected for the above unknown base sequence are located on a substrate in such a manner that the probes are isolated from one another;

(b) reacting a fluorescent-labeled single-stranded nucleic acid having a base sequence fully complementary to the base sequence of the first single-stranded nucleic acid probe with the probe array under conditions that mutually complementary single-stranded nucleic acids form a double-stranded nucleic acid, eliminating unreacted labelled single-stranded nucleic acids, then measuring the fluorescence intensity of each single-stranded nucleic acid probe on the above probe array, and obtaining a first template pattern showing a relationship between the probe positions on the probe array and fluorescence properties thereof;

(c) repeating the above step (b) for the remaining probes to obtain a second to nth template patterns each of which shows a relationship between the probe positions on the probe array and fluorescence properties thereof when one of single-stranded nucleic acid probes on the probe array forms a double-stranded nucleic acid with a single-stranded nucleic acid having a fully complementary base sequence thereto;

(d) reacting the probe array with a sample containing a target single-stranded nucleic acid under the same conditions as for obtaining the above template patterns, measuring the presence or absence and intensity of fluorescence from each single-stranded nucleic acid probe on the probe array to obtain a sample pattern showing a relationship between positions of single-stranded nucleic acid probes on the probe array and fluorescence properties thereof; and

(e) comparing the sample pattern with the first to nth template patterns obtained in the above steps (b) to (c), and, if there is a substantially identical template pattern, determining the unknown base sequence of the target single-stranded nucleic acid to be the base sequence of the single-stranded nucleic acid used for preparing the template pattern.

One technical feature of another embodiment of the present invention is that a threshold value is provided on fluorescence intensity to discriminate fluorescence positive and negative, e.g., to discriminate hybrids with one base mismatch from hybrids with two base mismatch. This embodiment is a method for determining an unknown base sequence in a given region of a target single-stranded nucleic acid, comprising the steps of (a) to (h):

(a) preparing a probe array in which a first to nth (n≧2) single-stranded nucleic acid probes having base sequences respectively complementary to every base sequences expected for the unknown base sequence are located on a substrate in such a manner that the probes are isolated from one another;

(b) reacting a first labeled single-stranded nucleic acid having a base sequence fully complementary to the first probe with the above probe array under conditions that complementary single-stranded nucleic acids form a double-stranded nucleic acid, measuring fluorescence from each probe on the above probe array to obtain a template pattern I showing a relationship between positions of the probes on the probe array and fluorescence properties thereof;

(c) analyzing the template pattern I to calculate an average fluorescent intensity (Fᵢ) for double-stranded nucleic acids having i mismatches;

(d) obtaining a difference (F_{1.0}) between the fluorescent intensity (F₀) of the fully complementary double-stranded nucleic acid having 0 mismatch and the average fluorescence intensity (F₁) of double-stranded nucleic acids having one mismatch, and further obtaining a difference (F_{i+1,i}) between the fluorescent intensity (Fᵢ₊₁) of double-stranded nucleic acids having (i+1) base mismatches and the fluorescent intensity (Fᵢ) of double-stranded nucleic acids having i base mismatches to determine i so as to be F_{i, i+1}<<F_{i-1, i};

(e) obtaining a template pattern II comprised of positive positions, where the positive positions correspond to probes that differ from the second probe in i or less bases, and negative positions to probes that differ from the second probe in more than i bases;

(f) repeating this step for all remaining probes to obtain template patterns III to n;

(g) reacting the above probe array with a sample containing a target single-stranded nucleic acid under the same condition as the condition of obtaining the template pattern I, measuring the presence or absence and intensity of fluorescence from the single-stranded nucleic acid probes on the above probe array to obtain a sample pattern showing a relationship between positions of single-stranded nucleic acid probes on the probe array and fluorescence properties thereof; and

(h) comparing the sample pattern with the template patterns I to n obtained in the above steps (b), (e) and (f), and if there is a template pattern substantially identical to the sample pattern, determining the unknown base sequence of the target single-stranded nucleic acid to be the base sequence of a single-stranded nucleic acid corresponding to the template pattern.

Employing this embodiment, patterns of spots identified as positive on the substrate are obtained as image, and the target sequence can be then analyzed by comparing the obtained pattern with predicted patterns and thereby unknown gene sequences can easily be determined.

Moreover, the present invention provides conditions for hybridization reaction to completely differentiate the one base mismatch from two base mismatches.

Furthermore, the hybridization reaction method of the present invention is characterized in that the step of reacting a sample containing a target single-stranded nucleic acid with a probe array comprises the steps of: heat-denaturing the probe array substrate in a solution containing the sample, lowering the temperature to a suitable temperature for a double-strand formation reaction while the substrate remains immersed in the sample solution, and carrying out the reaction in the sample solution.

In the above described method, the temperature for the heat denaturation is preferably 60° C. or higher. Further, the temperature for the double-strand formation reaction is 40° C. or higher. Still further, the time required for the heat denaturation is preferably 10 minutes or more.

The detection method of the present invention is a sample detection method using the above described hybridization reaction method, and is characterized in that washing is carried out at a raised temperature after the reaction step at a lowered temperature.

Furthermore, in the above method, it is preferable that the above described double-strand formation reaction is carried out in a sample solution of a high salt concentration, and the above described washing is carried out at a low salt concentration. What is more, it is preferable that the solution in the above double-stand formation reaction contains formamide.

### BRIEF DESCRIPTION OF THE DRAWINGS

FIG. 1 shows a disposition example with 64 types of probes;

FIG. 2 shows a pattern of would-be positive spots with a target nucleic acid;

FIG. 3 shows a pattern of would-be positive spots with a target nucleic acid having a mutant sequence;

FIG. 4 shows a pattern of fluorescence intensities obtained in Example 1;

FIG. 5 shows a predicted pattern in Example 2;

FIG. 6 shows a pattern of fluorescence intensities obtained in Example 2 with a threshold of 10%;

FIG. 7 shows a pattern of fluorescence intensities obtained in Example 3; and

FIG. 8 shows results of hybridization reaction using genomic DNA.

### DESCRIPTION OF THE PREFERRED EMBODIMENTS

The present invention is explained in detail.

<Call Using Fluorescence Image>

One embodiment of the present invention particularly effective when bases which may cause mismatching exist close to each other. Herein, this will be explained using 5′GATGGGNCTCNNGTTCAT3′ as an example, this sequence includes a base sequence corresponding to the 248th and 249th amino acids of tumor suppressor gene p53. This example is only to explain this invention roughly, not to limit the present invention to a specific array form or probe arrangement. The concept of the present invention that the results are processed as an image is applicable to any form of arrays. The SBH method is naturally subjected to the analysis of the present invention.

In the above example, when a full set of probes is prepared by replacing the base represented by N with any of four bases (A, G, C, T), that is, when three bases (no need for continuity) are examined, 4³=64 probes are arranged on the substrate. 4⁵=1024 probes are required to examine five bases.

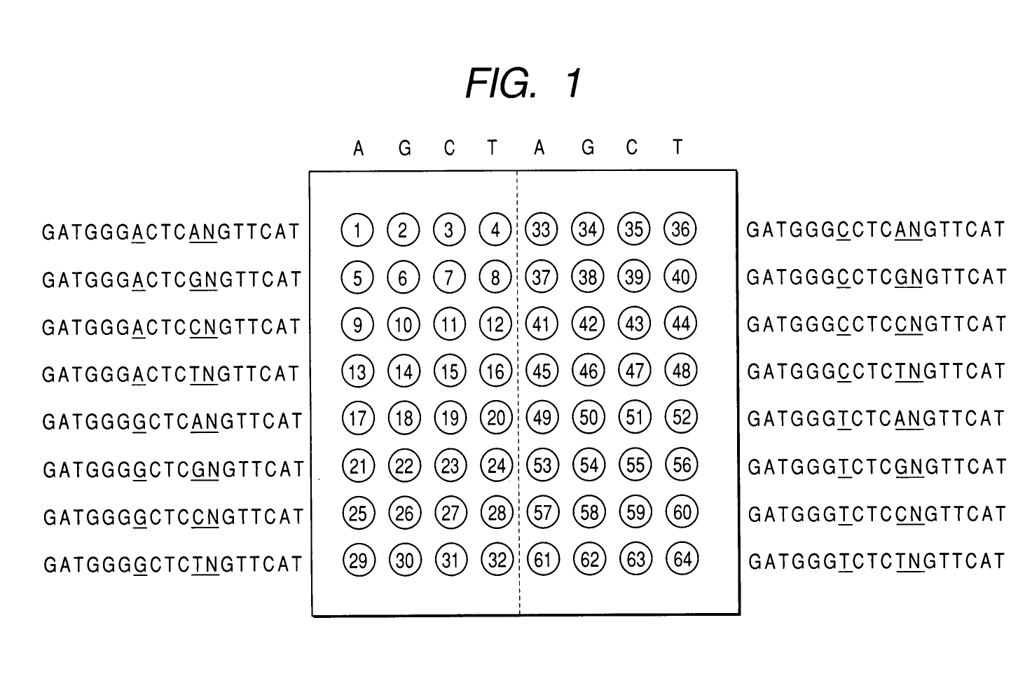

An example of the arrangement when 64 types of probes are used is shown in FIG. 1.

In this example, in the upper left quarter region of the array of 64 probes, are arranged the probes of which first N is A (probe number: 1-16), while in the lower left quarter region, the probes of which first N is G (probe number: 17-32). Similarly, in the upper right quarter region, probes of which first N is C (probe number: 33-48) are arranged and, in the lower right region, those having the first N of T (probe number: 49-64). In each region, the probes having the second N of A are positioned in the first column from the left, G, C and T for the second, third and fourth columns respectively. Also, probes having the third N of A are positioned in the first row from the top in each region, G, C and T in the second, third and fourth rows respectively. As a result, for example, the sequence of 5′GATGGGACTCAAGTTCAT3′ corresponds to the upper left corner spot. A target nucleic acid being 5′ATGAACCGGAGGCCCATC3′, which corresponds to the wild type gene, is expected to form a hybrid with a probe DNA 5′GATGGGCCTCCGGTTCAT3′ which is positioned at the crosspoint of the third column from the right and the third row from the top.

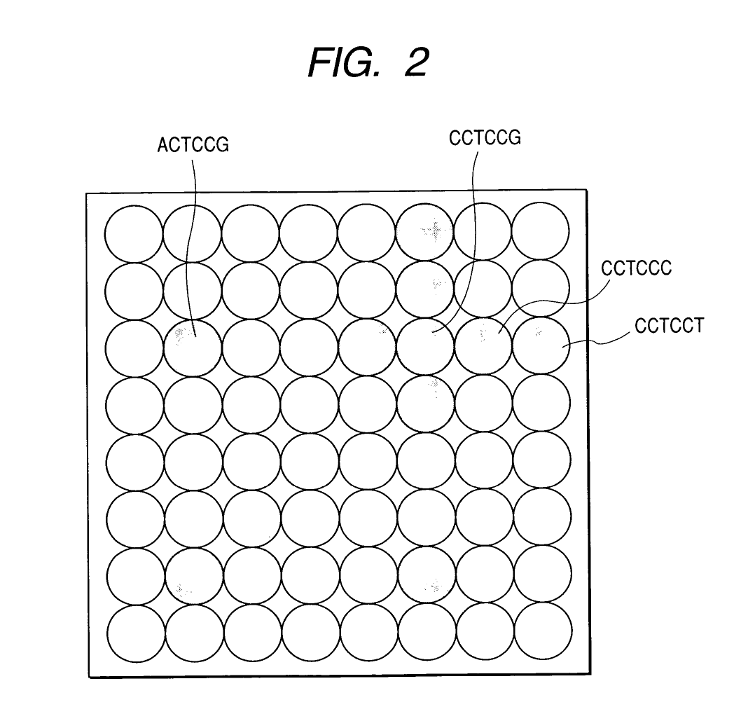

Now the case where one-base mismatch hybrids are treated as positive spots will be explained. In this case, if the fully matching sequence is the probe 42 (wild type), one-base mismatching sequences to be called positive correspond to 9 points (shadowed circles), forming a pattern together with the perfect match point as shown in FIG. 2.

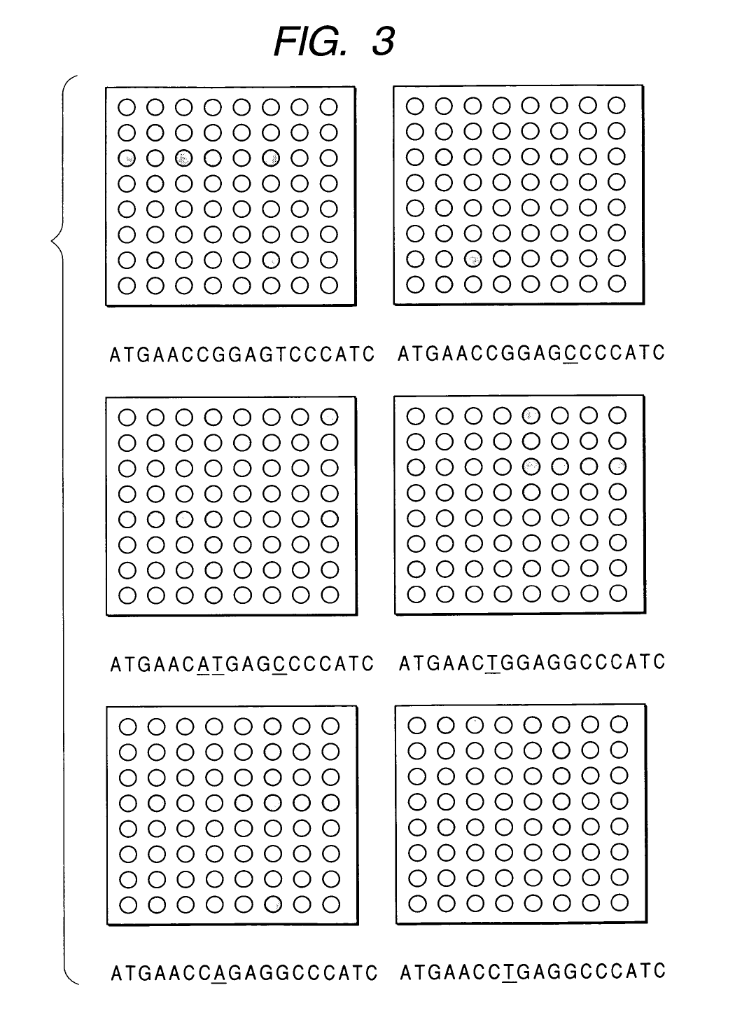

On the other hand, the pattern change will be observed with a target nucleic acid having a variant sequence to be identified, as shown in FIG. 3.

In the present invention, images of the expected fluorescent patterns composed of such full match and one-base mismatch hybrids are input into a computer memory device or the like in beforehand, and the call is performed by comparing a fluorescent image obtained by a predetermined method with the memory. Herein, detailed quantitative data of the fluorescence intensity of positive spots are not required. Simple judgement on whether the fluorescence is stronger than the threshold value which has been determined experimentally enables simple and automatic calling using a computer etc.

<Setting of Threshold>

When a probe of about 18 mer is used, the threshold is preferably set between the fluorescence intensity of the one-base mismatch and that of the two-base mismatch. Although the fluorescence intensity depends on the sequence or the reaction conditions, 50% to 25%, more preferably 30% to 20%, of the highest fluorescent intensity (normally of the full match hybrid) may be used as the threshold. When the length of the probe is shorter, the threshold must be lower.

Fluorescence of those having three-base mismatch will be below 10% of the maximum fluorescence, allowing complete discrimination.

A more specific calling method will be described with the above example.

When the hybridization reaction is carried out very selectively, strong fluorescence appears only at one point (the full match). When the sensitivity is increased gradually or the stringency in reaction conditions is reduced, as expected from FIG. 3 in the above-arranged example, the one-base mismatch points will appear in a row and a column crossing at the full match point. However, the actual fluorescent image is not always such that three spots each align in a row and column around a strong fluorescent point. Since six points not always have similar fluorescence intensity due to the hybrid stability difference, not all of the spots can be detected. However, at least some spots would be seen on those lines. At the same time, the remaining one-base mismatches may fluoresce at the expected positions, although the intensity might vary.

Sometimes, the full match hybrid and one-base mismatch hybrids may have similar fluorescence intensity to give a pattern consisting of the expected 10 spots of the full match and one-base mismatches.

Although the fluorescence intensity of two-base mismatch hybrids sometimes exceeds the threshold, they can be distinguished easily because of the divergence from the expected pattern.

Thus, the method of the present invention where calling is performed by comparing the expected pattern with the actually obtained fluorescent image has a feature that the presence or absence of variation in the test gene can be easily determined and, at the same time, the nature of the variation (which base(s) is changed to what base(s)) can be determined.

Further, when the result of hybridization using 64 probes is assessed, the idea of pattern assessment has an advantage that calling is more reliable than with only one spot. Since the hybrids with 64 DNA probes differ in heat stability between individual sequences, it is not ensured that the full match hybrid is always far more stable and radiates stronger fluorescence. In addition, it is often impossible to determine the strongest and full match spot due to the foreign matters on the substrate or the artifacts during the hybridization reaction. In this point, calling by pattern can compensate certain variation of fluorescence intensity, if any.

<Probe Length>

The probe length used for the present invention is approximately 8 mer to 30 mer, more preferably 12 mer to 25 mer. When it is shorter than 8 mer, stability of the hybrids having one-base mismatch is low and the fluorescence from the full match hybrid is superior, while when it is longer than 30 mer, the fluorescence of two-base mismatch hybrids may be stronger than that of one-base mismatches, for example, when mismatches locate at the both ends.

<Conditions of Hybridization Reaction>

Preferable hybridization conditions are: A substrate is soaked completely in a sample solution and heated for heat-denaturing both the DNA probes on the substrate and the sample DNA, then the substrate and the solution are cooled down gradually to perform the hybridization reaction at a rather high temperature. The salt concentration of the reaction mixture is desirably below 100 mM.

Appropriate temperature for heat denaturation is 60° C. or higher, preferably 80° C. or higher. The temperature for heat denaturation is determined depending on the stability of the substrate itself, length and concentration of the test DNA, type of the labeling compound. For example, with such a substrate prepared by binding DNA to a resin layer formed on the surface of the substrate, sometimes the resin layer is destroyed by heating at a high temperature. On the other hand, substrates prepared using a silane coupling agent are rather heat-stable and can be heated to a higher temperature. When the test DNA is a single-stranded DNA, the intramolecular double-stranded structure is melt at 70° C. or more, while when the sample is a double-stranded DNA or long single-stranded DNA, it is necessary to melt the double-stranded structure by heating at a higher temperature or by adding a denaturing agent such as formamide. Time required for heat denaturation is 10 min or more, preferably, about 30 min, depending on the microassay size and the volume of the sample solution.

The hybridization conditions are determined according to the conventional method where temperature and salt concentration are changed considering the length and sequence of the probes, and the type of the test sample. The suitable conditions for discriminating extremely similar sequences as in the present invention are 45° C. for over 3 hours in a solution containing 100 mM of sodium chloride. However, as the reaction time is greatly affected by the sample concentration, it is not limited to the above reaction conditions. With a sample of high concentration, calling within 3 hours is possible, while with a dilute sample, 10 hours or more of the reaction time is required. When formamide is added, the concentration of sodium chloride should be increased.

<Preparation of DNA Array>

How to prepare the DNA array suitable for the hybridization reaction of the present invention is exemplified below. However, since the purpose of the present invention is to provide a simple method for evaluating the hybridization pattern on the substrate to determine the base sequence of a sample, the substrate preparation method is not specifically limited.

DNA probes may be covalently bonded to the substrate by reacting the probes with functional groups on the substrate. The following is a method of coupling reaction between a maleimide group on the glass surface with an SH group at the end of DNA.

Maleimide groups can be incorporated onto the surface of a glass substrate, first, by introducing amino groups with an amino silane coupler onto the substrate, and then reacting the amino groups with a reagent containing N-(6-maleimidocaproyloxy)succinimide (EMCS reagent: Dojin Co., Ltd.). Introduction of an SH group to DNA can be performed by use of 5′-Thiol-Modifier C6 (Glen Research Company) on a DNA-automatic synthesizer.

Spots of the DNA probes are formed by the ink jet method on the substrate, then the probe DNA is fixed by the reaction between the maleimide groups on the substrate and the SH groups at the end of the DNA.

A DNA solution suitable for ink jet ejection to the maleimide-substrate is one containing glycerin, urea, thiodiglycol or ethylene glycol, acetylenol EH (Kawamura Fine Chemical Company-made) and isopropyl alcohol. Particularly, a solution containing 7.5% of glycerin, 7.5% of urea, 7.5% of thiodiglycol and 1% of acetylenol EH is preferable.

The array substrate to which DNA has been bonded is then soaked in an aqueous solution of 2% bovine serum albumin for 2 hours for blocking. Now it is ready for a hybridization reaction.

### ESAMPLES

The invention will be described in the following Examples in more detail.

### Example 1: Pattern Recognition I

1. Probe Design

It is well known that in the base sequence CGGAGG corresponding to the AA248 and AA249 of the tumor suppressor gene p53, frequently observed variation is those the first C to T, the second A to G for AA248, and the third G to T for AA249. Accordingly, aiming at these three positions, 64 types of probes were designed.

That is, the designed nucleic acid are 18-mer nucleic acids harboring variegated above mentioned six bases flanked by the common sequences, to be represented by 5′ATGAACNNGAGNCCCATC3′ where N corresponds to any of 4 bases, A, G, C and T. Actual probes to detect the above sequence should be have a complementary sequence of 5′GATGGGNCTCNNGTTCAT3′.

FIG. 1 shows an arrangement of 64 types of DNA probes on a substrate. Each sequence (SEQ ID NOs: 1 to 64) is specifically shown in Table 1.

TABLE 1

| | |
|---|---|
| SEQ ID NO. | Sequence |

| | |
|---|---|
| 1 | GATGGGACTCAAGTTCAT |
| 2 | GATGGGACTCAGGTTCAT |
| 3 | GATGGGACTCACGTTCAT |
| 4 | GATGGGACTCATGTTCAT |
| 5 | GATGGGACTCGAGTTCAT |
| 6 | GATGGGACTCGGGTTCAT |
| 7 | GATGGGACTCGCGTTCAT |
| 8 | GATGGGACTCGTGTTCAT |
| 9 | GATGGGACTCCAGTTCAT |
| 10 | GATGGGACTCCGGTTCAT |
| 11 | GATGGGACTCCCGTTCAT |
| 12 | GATGGGACTCCTGTTCAT |
| 13 | GATGGGACTCTAGTTCAT |
| 14 | GATGGGACTCTGGTTCAT |
| 15 | GATGGGACTCTCGTTCAT |
| 16 | GATGGGACTCTTGTTCAT |
| 17 | GATGGGGCTCAAGTTCAT |
| 18 | GATGGGGCTCAGGTTCAT |
| 19 | GATGGGGCTCACGTTCAT |
| 20 | GATGGGGCTCATGTTCAT |
| 21 | GATGGGGCTCGAGTTCAT |
| 22 | GATGGGGCTCGGGTTCAT |
| 23 | GATGGGGCTCGCGTTCAT |
| 24 | GATGGGGCTCGTGTTCAT |
| 25 | GATGGGGCTCCAGTTCAT |
| 26 | GATGGGGCTCCGGTTCAT |
| 27 | GATGGGGCTCCCGTTCAT |
| 28 | GATGGGGCTCCTGTTCAT |
| 29 | GATGGGGCTCTAGTTCAT |
| 30 | GATGGGGCTCTGGTTCAT |
| 31 | GATGGGGCTCTCGTTCAT |
| 32 | GATGGGGCTCTTGTTCAT |
| 33 | GATGGGCCTCAAGTTCAT |
| 34 | GATGGGCCTCAGGTTCAT |
| 35 | GATGGGCCTCACGTTCAT |
| 36 | GATGGGCCTCATGTTCAT |
| 37 | GATGGGCCTCGAGTTCAT |
| 38 | GATGGGCCTCGGGTTCAT |
| 39 | GATGGGCCTCGCGTTCAT |
| 40 | GATGGGCCTCGTGTTCAT |
| 41 | GATGGGCCTCCAGTTCAT |
| 42 | GATGGGCCTCCGGTTCAT |
| 43 | GATGGGCCTCCCGTTCAT |
| 44 | GATGGGCCTCCTGTTCAT |
| 45 | GATGGGCCTCTAGTTCAT |
| 46 | GATGGGCCTCTGGTTCAT |
| 47 | GATGGGCCTCTCGTTCAT |
| 48 | GATGGGCCTCTTGTTCAT |
| 49 | GATGGGTCTCAAGTTCAT |
| 50 | GATGGGTCTCAGGTTCAT |
| 51 | GATGGGTCTCACGTTCAT |
| 52 | GATGGGTCTCATGTTCAT |
| 53 | GATGGGTCTCGAGTTCAT |
| 54 | GATGGGTCTCGGGTTCAT |
| 55 | GATGGGTCTCGCGTTCAT |
| 56 | GATGGGTCTCGTGTTCAT |
| 57 | GATGGGTCTCCAGTTCAT |
| 58 | GATGGGTCTCCGGTTCAT |
| 59 | GATGGGTCTCCCGTTCAT |
| 60 | GATGGGTCTCCTGTTCAT |
| 61 | GATGGGTCTCTAGTTCAT |
| 62 | GATGGGTCTCTGGTTCAT |
| 63 | GATGGGTCTCTCGTTCAT |
| 64 | GATGGGTCTCTTGTTCAT |

5′ATGAACCGGAGGCCCATC3′ which is the sequence corresponding to the wild type gene is expected to form a hybrid with the DNA probe 42 of 5′GATGGGCCTCCGGTTCAT3′ located at the third point from the right and from the top.

In experiment of 64 hybrid formations, fluorescence from the one-base mismatch hybrids is also expected in addition to that from the full match hybrid, an expected pattern of the fluorescence from the full match hybrid and one-base mismatch hybrids is shown in FIG. 2.

2. Preparation of Substrate Introduced with Maleimide Group

<Substrate Cleaning>

A glass plate of 1-inch square was placed in a rack and soaked in an ultrasonic cleaning detergent overnight. Then, after 20 min of ultrasonic cleaning, the detergent was removed by washing with water. After rinsing the plate with distilled water, ultrasonic treatment was repeated in a container filled with distilled water, for additional 20 min. Then the plate was soaked in a prewarmed 1N sodium hydroxide solution for 10 min, washed with water and then distilled water.

<Surface Treatment>

Then the plate was soaked in an aqueous solution of 1% silane coupling agent (product of Shin-Etsu Chemical Industry: Trade name KBM 603) at a room temperature for 20 min, thereafter nitrogen gas was blown on the both sides blowing off water to dryness. The silane coupling treatment was completed by baking the plate in an oven at 120° C. for 1 hour. Subsequently, 2.7 mg of EMCS (N-(6-maleimidocaproyloxy)succinimide: Dojin Company) was weighed and dissolved in a 1:1 solution of DMSO/ethanol (final concentration: 0.3 mg/ml). The glass substrate treated with the silane coupling agent was soaked in this EMCS solution for 2 hours to react the amino group of the silane coupling agent with the carboxyl group of EMCS. At this stage, the maleimide group of EMCS is transferred to the glass surface. After that, the glass plate was washed with distilled water, and dried with nitrogen gas to be used for a coupling reaction with DNA.

3. Coupling of DNA to the Substrate

<Synthesis of 64 DNA Probes>

The above 64 types of probe DNAs each having an SH group (thiol group) at the 5′ terminus were synthesized by Becks Co., Ltd. at our request.

Ejection of DNA Probes

The above 64 types of DNAs were ejected respectively as follows. Each DNA was dissolved in water and diluted with SG Clear (aqueous solution containing 7.5% of glycerin, 7.5% of urea, 7.5% of thiodiglycol and 1% of acetylenol EH), to a final concentration of 8 μM. Then 100 μl of this DNA solution was filled into a nozzle of a BJ printer Head BC 62 (Canon) modified to eject a small amount, and to eject six solutions per head. Two heads were used at a time so that 12 types of DNAs could be ejected at once, and the heads were changed 6 times so that 64 spots of 64 types of DNAs were formed on the glass plates independently.

Sixty-four probes were spotted with a diameter of 70 μm and a pitch of 200 μm to form a matrix of 8×8. After that, the plate was left standing in a humidified chamber for 30 min for linking reaction of the probe DNA to the substrate.

Hybridization Reaction

<Blocking Reaction>

After completion of the reaction, the substrate was washed with a 1 M NaCl/50 mM phosphate buffer solution (pH 7.0) to wash out thoroughly the DNA solution on the glass surface. Then, this was soaked in an aqueous solution of 2% bovine serum albumin and allowed to stand for 2 hours to carry out a blocking reaction.

<Preparation of Model Sample DNA>

Rhodamine labeled DNA No. 1 of the same length as the probes but having the wild type sequence of p53 gene was prepared. The sequence is shown below and rhodamine is bonded to the 5′ terminus.

No. 1 : 5′Rho-ATGAACCGGAGGCCCATC3′ (SEQ ID No: 65)

<Hybridization Conditions>

Two milliliters of a 10 nM model sample DNA solution containing 100 mM NaCl was applied to the DNA array substrate in a hybridization bag, and the bag was initially heated at 80° C. for 10 min. Then the temperature of the incubator was lowered to 45° C. and the reaction was continued for 15 hours.

5. Detection

<Detection Method>

The detection was performed by connecting an image analysis processing apparatus, ARGUS (a product of Hamamatsu Photonics) to a fluorescence microscope (a product of Nicon).

<Result>

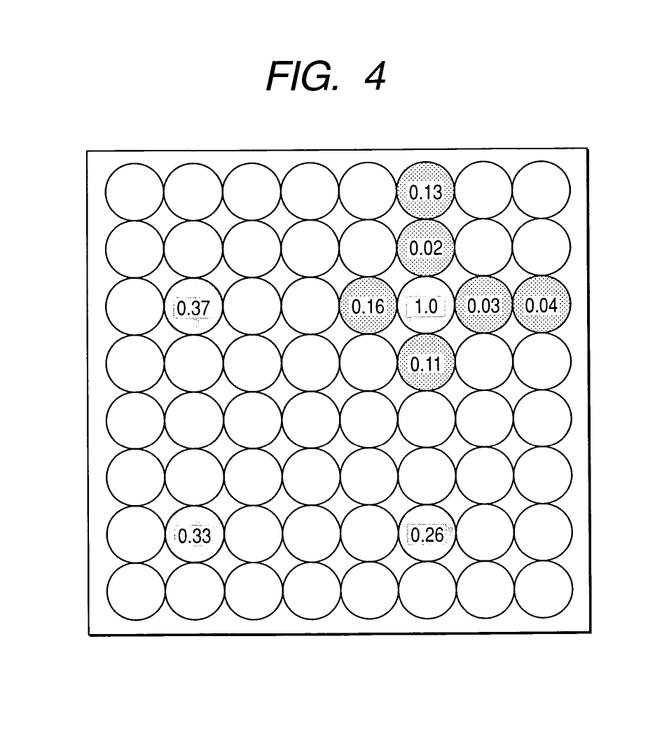

The fluorescence intensities obtained from the model hybridization reaction with the labeled DNA No. 1 (18-mer) are shown in FIG. 4. The maximum value of the fluorescence intensity was obtained at the spot of probe 42 which is fully complementary to DNA No. 1. Taking this intensity as the maximum value (1.0), the threshold is set at 20% of this value and the spots having higher intensity are painted dark.

The spots of probes 10, 26 and 58 of one-base mismatch hybrids have fluorescence higher than the threshold, and it is understood that the location is well coincident with FIG. 2 of the expected pattern. By lowering the threshold further, in addition to the above 5 spots, the spots of other one-base mismatch probes appeared around the full matching probe in vertical and horizontal lines, in coincidence with the expected pattern.

### Example 2: Pattern Recognition II

A DNA array of 64 types of probes was prepared in the same manner as in Example 1, and the hybridization reaction was performed using a rhodamine-labeled DNA No. 2 as a model sample. The DNA No. 2 has a sequence complementary to the No. 46 probe of FIG. 1.

No. 2: 5′Rho-ATGAACCAGAGGCCCATC3′ (SEQ ID No: 66)

The reaction conditions of hybridization are the same as in Example 1.

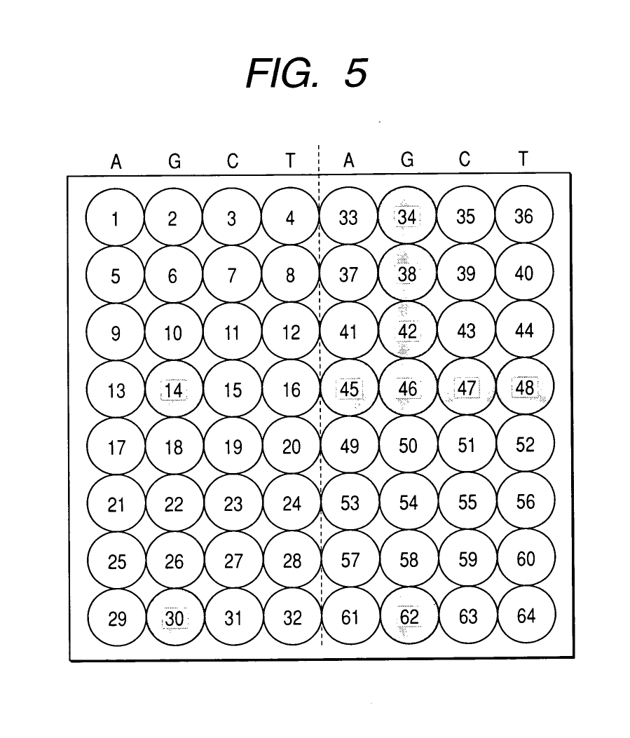

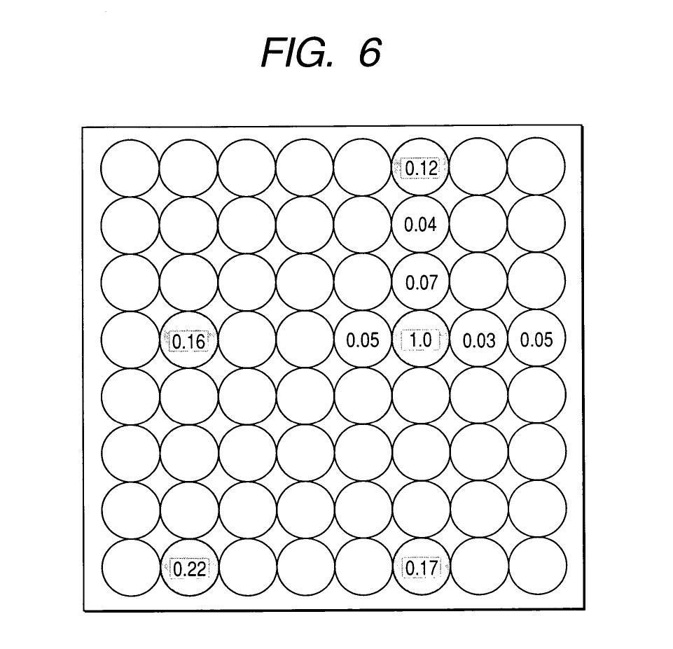

FIG. 5 is an expected pattern consisting of the perfect match and one-base mismatch hybrids, and the resulted pattern obtained as in Example 1 is shown in FIG. 6. The threshold is set at 10% of the maximum value. When the detected spots are painted dark, the result is well corresponding to the expectation.

### Example 3: Pattern Recognition III

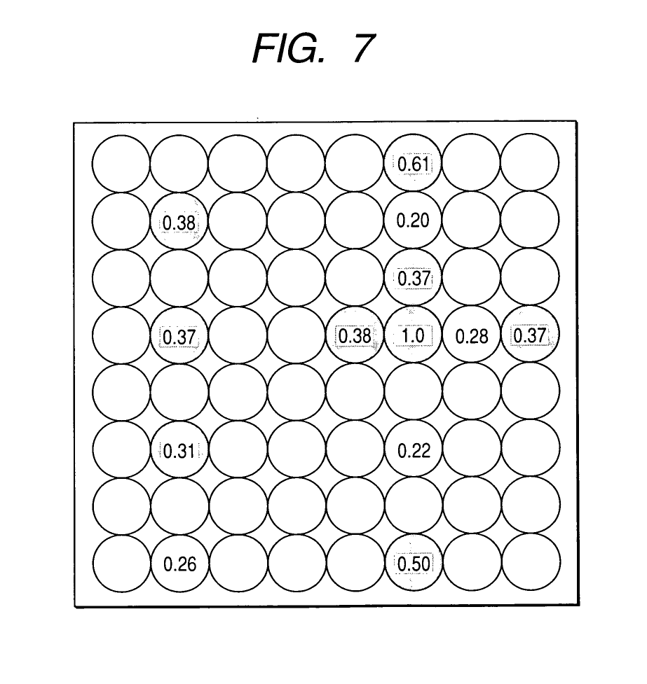

An experiment was carried out in the same manner as in Example 2 except that the concentration of the sample DNA used for the hybridization reaction was 5 nM and the reaction was carried out at 40° C. overnight. The result obtained is shown in FIG. 7.

If the threshold is set as 50%, fluorescence was detected at the positions (shaded parts) of Nos. 34 and 62 probes (one-base mismatch) in addition to No. 46 (full match), and with further reduction of the threshold to 30%, the result was coincident with the expected pattern. In this case, Nos. 6, 22 and 54 of two-base mismatch probes were detected, but the two-base mismatch can be distinguished from the one-base mismatch as the deviation from the expected pattern of one-base mismatch, and No. 46 can be called as the full matched probe.

### Example 4 (Preparation of Genomic Sample DNA HSC5)

The process of probe design to blocking reaction was carried out in the same manner as in Example 1 to obtain a DNA array substrate for determination. Using this DNA array substrate, the following operation was carried out.

1) Amplification of Exons of p53 Gene of HSC5

Based on the base sequences flanking introns, the following PCR primers were synthesized.

(SEQ ID NO: 67)

| | | |
|---|---|---|
| E5S: | 5′-TGTTCACTTGTGCCCTGACT-3′ | (exon 5, sense) |

(SEQ ID NO: 68)

| | | |
|---|---|---|
| E5A: | 5′-TGAGGAATCAGAGGCCTGG-3′ | (exon 5, antisense) |

(SEQ ID NO: 69)

| | | |
|---|---|---|
| E6S: | 5′-GCCTCTGATTCCTCACTGAT-3′ | (exon 6, sense) |

(SEQ ID NO: 70)

| | | |
|---|---|---|
| E6A: | 5′-TTAACCCCTCCTCCCAGAGA-3′ | (exon 6, antisense) |

(SEQ ID NO: 71)

| | | |
|---|---|---|
| E7S: | 5′-ACTGGCCTCACTTTGGGCCT-3′ | (exon 7, sense) |

(SEQ ID NO: 72)

| | | |
|---|---|---|
| E7A: | 5′-TGTGCAGGGTGGCAAGTGGC-3′ | (exon 7, antisense) |

(SEQ ID NO: 73)

| | | |
|---|---|---|
| E8S: | 5′-TAAATGGGACAGGTAGGACC-3′ | (exon 8, sense) |

(SEQ ID NO: 74)

| | | |
|---|---|---|
| E8A: | 5′-TCCACCGCTTCTTGTCCTGC-3′ | (exon 8, antisense) |

PCR reaction was carried out under such conditions that 10 to 25 ng of genomic DNA and each of the exon primer sets (0.4 μM) were added to 50 μL of a PCR reaction solution and a cycle of 94° C. (30 seconds) and 60° C. (45 seconds) was repeated 40 times.

The amplified products were 269, 181, 171 and 229-base long corresponding to exons 5 to 8, respectively.

2) Labeling of Exons

Tetramethyl rhodamine-labeled ssDNAs corresponding to the above four exons were prepared by PCR as follows: a cycle of 96° C. (30 seconds), 50° C. (30 seconds) and 60° C. (4 minutes) was repeated 25 times using the respective amplified exon DNA as a template, 0.2 μM of corresponding sense primer and 10 μM tetramethyl rhodamine-labelled dUTP (Fluoro Red, Amersham Pharmacia Biotech).

The obtained single-stranded DNAs were purified by gel filtration.

3) Hybridization Reaction with Labelled Exon

The above obtained tetramethyl rhodamine labelled ssDNA was dissolved in a 6×SSPE solution (0.9 M NaCl, 60 μM NaH₂PO₄, 6 μM EDTA) containing 20% formamide, and 2 mL of the solution was poured into a bag containing a DNA array substrate for hybridization reaction. After heating at 80° C. for 2 to 10 minutes, the temperature of the incubator was reduced and kept to 45° C. for 15 hours for reaction.

Thereafter, the above DNA array was immersed in a 2×SSPE solution (0.3 M NaCl, 20 μM NaH₂PO₄, 2 μM EDTA), and the temperature was raised to 55° C. to carry out washing.

<Detection>

A detection operation was carried out in the same manner as in Example 1.

<Result>

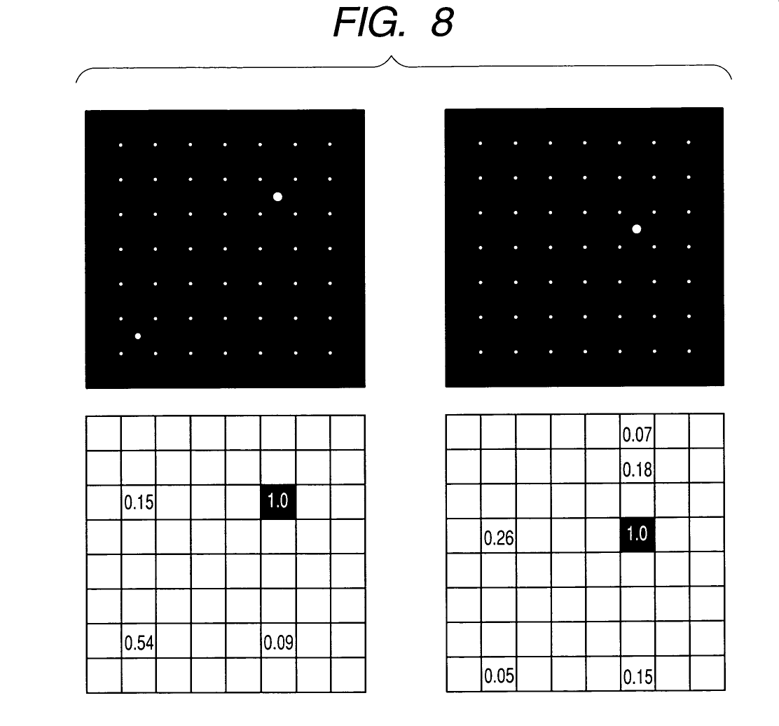

Spots of Nos. 10, 26 and 58 emitted fluorescence, and it was shown that the predicted pattern and the obtained pattern (FIG. 8) are in a match.

### Example 5 (Detection of p53 Gene of HSC4)

A DNA array substrate comprised of 64 types of probes was obtained in the same manner as in Example 1. Then, a hybridization reaction was carried out in the same manner as in Example 2 with the exception that, instead of the rhodamine labelled DNA, HSC4 DNA containing sequence No. 2 was used as a model sample. Reaction conditions were the same as in Example 4. As a result, fluorescence was observed at position No. 14, and the obtained pattern matched well with the predicted pattern.

Thus, when compared with the previous methods for making determination only by the presence or absence of a hybrid, the method of the present invention enables to perform detection with good precision by taking the fluorescent intensity of one base mismatch into consideration.

Since hybrids obtained by hybridization with DNA probes have a different heat stability depending on the sequence, there is no guarantee that a hybrid of perfect match is always overwhelmingly stable and emits strong fluorescence. The determination by a pattern has an advantage that it allows a more reliable determination than the determination with only one spot.

Due to dust on the substrate or artifact generated during hybridization reaction, it is often impossible to determine the strongest thus perfectly matched spot. However, even when fluorescent intensity somewhat varies, determination with pattern of the present invention enables to complement it.

Therefore, according to the present invention, there is provided a test method which enables a simple and efficient screening for gene variations.

1. A method for performing a hybridization reaction comprising the steps of:
providing a sample comprising a target single-stranded nucleic acid and a probe array;
heat-denaturing the probe array in a solution containing the sample; and
reducing temperature to the extent suitable for a double-strand formation reaction,
wherein the probe array remains immersed in the sample solution during reducing the temperature.

2. The hybridization reaction method according to claim 1, wherein the temperature of said heat denaturation is 60° C. or higher.

3. The hybridization reaction method according to claim 1, wherein the temperature of said double-strand formation reaction is 40° C. or higher.

4. The hybridization reaction method according to claim 1, wherein the time required for said heat denaturation is 10 minutes or more.

5. A method for detecting a sample by using a hybridization reaction method according to claim 1, wherein after the step of lowering temperature to perform a reaction, washing is carried out at a raised temperature.

6. The detection method according to claim 5, wherein said double-strand formation reaction is carried out with a high salt concentration, and said washing is carried out with a low salt concentration.

7. The detection method according to any one of claims 1 to claim 6, wherein the solution in said double-strand formation reaction contains formamide.

8. The hybridization reaction method according to claim 1, wherein the probe array comprises a substrate and a single-stranded nucleic acid probe fixed on the substrate, said nucleic acid probe being capable of specifically forming a hybrid with the target single-stranded nucleic acid.

9. The hybridization reaction method according to claim 8, wherein the probe array comprises different single-stranded nucleic acid probes fixed on the substrate.


SEQUENCE LISTING (74 sequences)

SEQ ID NO: 1 - DNA, 18 bp, Homo sapiens
gatgggactcaagttcat

SEQ ID NO: 2 - DNA, 18 bp, Homo sapiens
gatgggactcaggttcat

SEQ ID NO: 3 - DNA, 18 bp, Homo sapiens
gatgggactcaggttcat

SEQ ID NO: 4 - DNA, 18 bp, Homo sapiens
gatgggactcatgttcat

SEQ ID NO: 5 - DNA, 18 bp, Homo sapiens
gatgggactcgagttcat

SEQ ID NO: 6 - DNA, 18 bp, Homo sapiens
gatgggactcgggttcat

SEQ ID NO: 7 - DNA, 18 bp, Homo sapiens
gatgggactcgcgttcat

SEQ ID NO: 8 - DNA, 18 bp, Homo sapiens
gatgggactcgtgttcat

SEQ ID NO: 9 - DNA, 18 bp, Homo sapiens
gatgggactccagttcat

SEQ ID NO: 10 - DNA, 18 bp, Homo sapiens
gatgggactccggttcat

SEQ ID NO: 11 - DNA, 18 bp, Homo sapiens
gatgggactcccgttcat

SEQ ID NO: 12 - DNA, 18 bp, Homo sapiens
gatgggactcctgttcat

SEQ ID NO: 13 - DNA, 18 bp, Homo sapiens
gatgggactctagttcat

SEQ ID NO: 14 - DNA, 18 bp, Homo sapiens
gatgggactctggttcat

SEQ ID NO: 15 - DNA, 18 bp, Homo sapiens
gatgggactctcgttcat

SEQ ID NO: 16 - DNA, 18 bp, Homo sapiens
gatgggactcttgttcat

SEQ ID NO: 17 - DNA, 18 bp, Homo sapiens
gatggggctcaagttcat

SEQ ID NO: 18 - DNA, 18 bp, Homo sapiens
gatggggctcaggttcat

SEQ ID NO: 19 - DNA, 18 bp, Homo sapiens
gatggggctcacgttcat

SEQ ID NO: 20 - DNA, 18 bp, Homo sapiens
gatggggctcatgttcat

SEQ ID NO: 21 - DNA, 18 bp, Homo sapiens
gatggggctcgagttcat

SEQ ID NO: 22 - DNA, 18 bp, Homo sapiens
gatggggctcgggttcat

SEQ ID NO: 23 - DNA, 18 bp, Homo sapiens
gatggggctcgcgttcat

SEQ ID NO: 24 - DNA, 18 bp, Homo sapiens
gatggggctcgtgttcat

SEQ ID NO: 25 - DNA, 18 bp, Homo sapiens
gatggggctccagttcat

SEQ ID NO: 26 - DNA, 18 bp, Homo sapiens
gatggggctccggttcat

SEQ ID NO: 27 - DNA, 18 bp, Homo sapiens
gatggggctcccgttcat

SEQ ID NO: 28 - DNA, 18 bp, Homo sapiens
gatggggctcctgttcat

SEQ ID NO: 29 - DNA, 18 bp, Homo sapiens
gatggggctctagttcat

SEQ ID NO: 30 - DNA, 18 bp, Homo sapiens
gatggggctctggttcat

SEQ ID NO: 31 - DNA, 18 bp, Homo sapiens
gatggggctctcgttcat

SEQ ID NO: 32 - DNA, 18 bp, Homo sapiens
gatggggctcttgttcat

SEQ ID NO: 33 - DNA, 18 bp, Homo sapiens
gatgggcctcaagttcat

SEQ ID NO: 34 - DNA, 18 bp, Homo sapiens
gatgggcctcaggttcat

SEQ ID NO: 35 - DNA, 18 bp, Homo sapiens
gatgggcctcacgttcat

SEQ ID NO: 36 - DNA, 18 bp, Homo sapiens
gatgggcctcacgttcat

SEQ ID NO: 37 - DNA, 18 bp, Homo sapiens
gatgggcctcgagttcat

SEQ ID NO: 38 - DNA, 18 bp, Homo sapiens
gatgggcctcgggttcat

SEQ ID NO: 39 - DNA, 18 bp, Homo sapiens
gatgggcctcgcgttcat

SEQ ID NO: 40 - DNA, 18 bp, Homo sapiens
gatgggcctcgtgttcat

SEQ ID NO: 41 - DNA, 18 bp, Homo sapiens
gatgggcctccagttcat

SEQ ID NO: 42 - DNA, 18 bp, Homo sapiens
gatgggcctccggttcat

SEQ ID NO: 43 - DNA, 18 bp, Homo sapiens
gatgggcctcccgttcat

SEQ ID NO: 44 - DNA, 18 bp, Homo sapiens
gatgggcctcctgttcat

SEQ ID NO: 45 - DNA, 18 bp, Homo sapiens
gatgggcctctagttcat

SEQ ID NO: 46 - DNA, 18 bp, Homo sapiens
gatgggcctctggttcat

SEQ ID NO: 47 - DNA, 18 bp, Homo sapiens
gatgggcctctcgttcat

SEQ ID NO: 48 - DNA, 18 bp, Homo sapiens
gatgggcctcttgttcat

SEQ ID NO: 49 - DNA, 18 bp, Homo sapiens
gatgggtctcaagttcat

SEQ ID NO: 50 - DNA, 18 bp, Homo sapiens
gatgggtctcaggttcat

SEQ ID NO: 51 - DNA, 18 bp, Homo sapiens
gatgggtctcacgttcat

SEQ ID NO: 52 - DNA, 18 bp, Homo sapiens
gatgggtctcatgttcat

SEQ ID NO: 53 - DNA, 18 bp, Homo sapiens
gatgggtctcgagttcat

SEQ ID NO: 54 - DNA, 18 bp, Homo sapiens
gatgggtctcgggttcat

SEQ ID NO: 55 - DNA, 18 bp, Homo sapiens
gatgggtctcgcgttcat

SEQ ID NO: 56 - DNA, 18 bp, Homo sapiens
gatgggtctcgtgttcat

SEQ ID NO: 57 - DNA, 18 bp, Homo sapiens
gatgggtctccagttcat

SEQ ID NO: 58 - DNA, 18 bp, Homo sapiens
gatgggtctccggttcat

SEQ ID NO: 59 - DNA, 18 bp, Homo sapiens
gatgggtctcccgttcat

SEQ ID NO: 60 - DNA, 18 bp, Homo sapiens
gatgggtctcctgttcat

SEQ ID NO: 61 - DNA, 18 bp, Homo sapiens
gatgggtctctagttcat

SEQ ID NO: 62 - DNA, 18 bp, Homo sapiens
gatgggtctctggttcat

SEQ ID NO: 63 - DNA, 18 bp, Homo sapiens
gatgggtctctcgttcat

SEQ ID NO: 64 - DNA, 18 bp, Homo sapiens
gatgggtctcttgttcat

SEQ ID NO: 65 - DNA, 18 bp, Homo sapiens
atgaaccggaggcccatc

SEQ ID NO: 66 - DNA, 18 bp, Homo sapiens
atgaaccagaggcccatc

SEQ ID NO: 67 - DNA, 20 bp, Homo sapiens
tgttcacttgtgccctgact

SEQ ID NO: 68 - DNA, 19 bp, Homo sapiens
tgaggaatcagaggcctgg

SEQ ID NO: 69 - DNA, 20 bp, Homo sapiens
gcctctgattcctcactgat

SEQ ID NO: 70 - DNA, 20 bp, Homo sapiens
ttaacccctcctcccagaga

SEQ ID NO: 71 - DNA, 20 bp, Homo sapiens
actggcctcactttgggcct

SEQ ID NO: 72 - DNA, 20 bp, Homo sapiens
tgtgcagggtggcaagtggc

SEQ ID NO: 73 - DNA, 20 bp, Homo sapiens
taaatgggacaggtaggacc

SEQ ID NO: 74 - DNA, 20 bp, Homo sapiens
tccaccgcttcttgtcctgc